(19) United States
(12) Patent Application Publication
(10) Pub. No.: US 2005/0096601 A1
(43) Pub. Date: May 5, 2005

(54) Syringe with integral safety system

(75) Inventor: Mark Christopher Doyle, San Diego, CA (US)

Correspondence Address: ORRICK, HERRINGTON & SUTCLIFFE, LLP, 4 PARK PLAZA, SUITE 1600, IRVINE, CA, 92614-2558, US

(73) Assignee: SAFETY SYRINGES, INC.

(21) Appl. No.: 11/009,195

(22) Filed: Dec. 10, 2004

Related U.S. Application Data
(62) Division of application No. 10/113,464, filed on Mar. 27, 2002.

(51) Int. Cl. A61M 5/32

(52) U.S. Cl. 604/198

(57) ABSTRACT

An injection device for delivery of a therapeutic agent is provided that includes a glass syringe with an integrally molded lug extending radially from the syringe and a guard slidably attached to the syringe. One set of detents on the guard and the lug of the syringe retain the guard in the retracted position. Another set of detents on the guard and the lug of the syringe retain the guard in the extended position. Another injection device for delivery of a therapeutic agent is provided that includes a glass syringe with an integrally molded disk that extends radially from the syringe and a guard slidably coupled to the syringe. One set of detents on the guard and the integrally molded disk of the syringe retain the guard in a retracted position. Another set of detents on the guard and the integrally molded disk retain the guard in an extended position.

### FIELD OF THE INVENTION

The present invention relates generally to injection devices for administering medical products to patients, particularly to safety devices for syringes, and more particularly to syringe devices that include a needle guard slidably coupled to a syringe for covering a needle of the syringe after use.

### BACKGROUND

Medication is often dispensed using a medicine cartridge, such as a syringe, having a barrel with a needle extending from one end and a plunger slidably inserted into the other end. Such cartridges are often referred to as “pre-filled syringes” because they may contain a specific dosage or volume of medication when they are initially provided. By comparison, conventional syringes are furnished empty and must be filled by the user prior to making an injection.

Alternatively, a medicine cartridge, such as an ampule or a vial, may be used that includes a penetrable seal instead of a needle on one end of the barrel, and/or a piston rather than a plunger on the other end. Such medicine cartridges are generally inserted into an adapter that includes a hollow body adapted to hold the cartridge, a plunger to engage and move the piston in the cartridge, and/or a double-ended needle to penetrate the seal and communicate with the interior of the barrel.

It is also desirable to have a syringe or cartridge wherein only a glass surface contacts the therapeutic agent. A glass surface is typically less likely to react with a therapeutic agent stored within the syringe. Glass is also generally easier to sterilize, such as, e.g., in an autoclave, than other materials such as plastics.

Because of the risk of communicable diseases, a number of syringes and adapters have been developed that are intended to prevent accidental needle sticks and/or inadvertent reuse of a syringe. Retractable needle devices have been suggested for this purpose that include a cartridge that allows a needle of the cartridge to be withdrawn into the barrel after medication is dispensed from it. For example, U.S. Pat. No. 4,973,316 issued to Dysarz discloses a syringe including a barrel having a needle assembly that is slidable within the barrel between an exposed position such that a needle on the assembly extends from the barrel and a retracted position wherein the needle assembly is withdrawn into the barrel. The needle assembly is initially locked in the exposed position, but may be disengaged upon depression of the plunger, whereupon a spring biases the assembly towards the retracted position, thereby withdrawing the needle into the barrel. The activation of the spring may, however, cause a sudden movement of the assembly that may surprise or result in discomfort to the patient. This may be the case, for example, when the assembly is forcibly, and quickly, biased against the patient's skin. The sudden movement of the assembly may also surprise a user who is unprepared for the quick movement of the assembly.

Alternatively, syringe holders have been suggested that include a body within which a conventional syringe or cartridge may be received, and a shield that is manually slidable with respect to the body to cover the needle. For example, U.S. Pat. No. 6,030,366, issued to Mitchell, which is assigned to the assignee of the present application, discloses a self-shielding guard that includes a body having an open proximal end for inserting a syringe into a cavity within the body, and a distal end with an opening through which a needle on the syringe may extend once received in the body. A shield is slidable over the body between retracted and extended positions to expose and cover the needle, respectively. With the shield in the retracted position and the needle exposed, an injection may be made, and then the shield may be manually advanced to the extended position. In the extended position, cooperating detents and detent pockets on the body and shield substantially permanently lock together, thereby preventing reuse of the needle, reducing the risk of accidental needle sticks, and/or facilitating disposal of the syringe.

Therefore, it is desirable to provide for an injection device wherein the syringe is formed from glass that also includes a slidable shield or guard for covering the needle after to use to prevent accidental needle pricks. Those in the art have also failed to provide for glass syringes with integrally molded detents that are resistant to breakage.

Accordingly, an injection device that includes a needle shield for covering a needle after use would be useful.

### SUMMARY OF THE INVENTION

The present invention is directed to injection devices for administering medical products, such as drugs, vaccines, or diagnostic or therapeutic agents, to patients. More particularly, the present invention is directed to safety devices for glass syringes, e.g., syringe devices that include a needle guard slidably coupled to a syringe for covering a needle of the syringe after use.

In accordance with a first aspect of the present invention, an injection device is provided that includes a syringe, preferably a glass syringe, having a proximal end, a distal end, and a plunger extending from the proximal end. A lug, which may be a pair of opposing lugs, are integrally formed on the syringe, and extend radially from the syringe proximal to the distal end. A guard is slidably attached to the syringe that includes a proximal end, a distal end, and a slot for receiving the lug of the syringe therein. The guard is movable from a first position, where the distal end of the syringe is exposed, to a second position, where the guard covers the distal end. A needle may extend from the distal end of the syringe. Additionally, the lug and the guard may include cooperating detents for retaining the guard in at least one of, and preferably each of, the first and second positions.

In a preferred embodiment, the lug may include distal and proximal outer edges that may be ramped, and a notch between the outer edges that is configured to receive one or more detents on the guard, thereby at least partially defining the cooperating detents. The guard may include one or more detents that extend into the slot; the detents may be received in the notch to retain the guard in the first and/or second positions. Preferably, the guard may include first detents extending into the slot adjacent the distal end of the guard and second detents extending into the slot adjacent the proximal end for securing the guard in the first and second positions, respectively.

In accordance with a second aspect of the present invention, an injection device is provided that includes a glass syringe having a body with proximal and distal ends, wherein the body of the syringe defines a cavity configured for receiving a medical product therein. A glass disk is integrally molded on the syringe, and preferably extends radially outwardly from the distal end. In one embodiment, the disk may have a diameter that is larger than a diameter of the body of the syringe.

A guard is slidably attached to the syringe. The guard includes a distal portion, and a proximal portion that is sized for sliding along the body of the syringe. In a preferred embodiment, the distal portion has a diameter larger than a diameter of the disk, and the proximal portion has a diameter smaller than the diameter of the disk. The guard may include a tapering intermediate portion that lies between the distal and proximal portions. The guard is slidable between a first position where the distal end of the syringe is exposed and a second position where the distal end is substantially covered by the guard. A needle may extend from the distal end of the syringe, or from a hub on the distal end of the syringe. Detents may be included on the guard for engaging the disk for retaining the guard in at least one of the first and second positions.

The detents may include one or more distal detents on an inner surface of the distal portion of the guard that may engage the disk to retain the guard in the first position, e.g., during administration of a medical product in the syringe to a patient. The injection device may also include one or more proximal detents proximal to the distal detents, e.g., on an inner surface of the distal portion of the guard adjacent the proximal portion. The proximal detents may engage the disk to retain the guard in the second position. Preferably, the proximal detents include substantially blunt proximal edges for preventing subsequent proximal movement of the guard from the second position towards the first position.

In accordance with a third aspect of the present invention, an injection device is provided that includes a glass syringe with a body having a proximal end, a distal end, a plunger extending from the proximal end, and a hub on the distal end. A glass disk is integrally molded to the syringe, and extends radially outwardly from the hub. A guard is slidably attached to the syringe. The guard includes a distal portion and a proximal portion, and is slidable between a first position and a second position. A first detent on an inner surface of the guard retains the guard in the first position. A second detent on an inner surface of the guard retains the guard in the second position. The distal end of the syringe may be exposed in the first position, and covered by the guard in the second position. The injection device may also include a needle that is coupled to the hub. The hub may include a connector to which the needle is attached.

Other objects and features of the present invention will become apparent from consideration of the following description taken in conjunction with the accompanying drawings.

### BRIEF DESCRIPTION OF THE DRAWINGS

For a better understanding of the invention, and to show how it may be carried into effect, reference will be made, by way of example, to the accompanying drawings.

FIG. 1A is a side view of a first preferred embodiment of an injection device, including a guard in a retracted position, in accordance with the present invention.

FIG. 1B is a side view of a first preferred embodiment of an injection device, including a guard in an extended position, in accordance with the present invention.

FIG. 2 is a top view of a syringe for use in the injection device shown in FIGS. 1A and 1B.

FIG. 3A is a top view of a guard for use in the injection device shown in FIGS. 1A and 1B.

FIG. 3B is a top view of another embodiment of a guard for use in the injection device shown in FIGS. 1A and 1B.

FIG. 3C is a top view of an alternative embodiment of a guard for use in the injection device shown in FIGS. 1A and 11B.

FIG. 4A is a side view of a second preferred embodiment of an injection device, including a guard in a retracted position, in accordance with the present invention.

FIG. 4B is a side view of a second preferred embodiment of an injection device, including a guard in an extended position, in accordance with the present invention.

FIG. 5 is a side view of a syringe for use in the injection device shown in FIGS. 4A and 4B.

FIG. 6 is a cross-sectional view of the syringe illustrated in FIG. 5, taken along the line 6-6.

FIG. 7 is a side view of a guard for use in the injection device shown in FIGS. 4A and 4B.

FIG. 8A is a cross-sectional view of the guard illustrated in FIG. 7, taken along the line 8A-8A.

FIG. 8B is a perspective cross-sectional view of the guard illustrated in FIG. 7.

FIG. 9 is an expanded side view of the distal portion of the injection device shown in FIGS. 4A and 4B.

FIG. 10 is an expanded side view of the injection device shown in FIGS. 4A and 4B, showing a disk on the syringe being retained by the guard.

### DETAILED DESCRIPTION

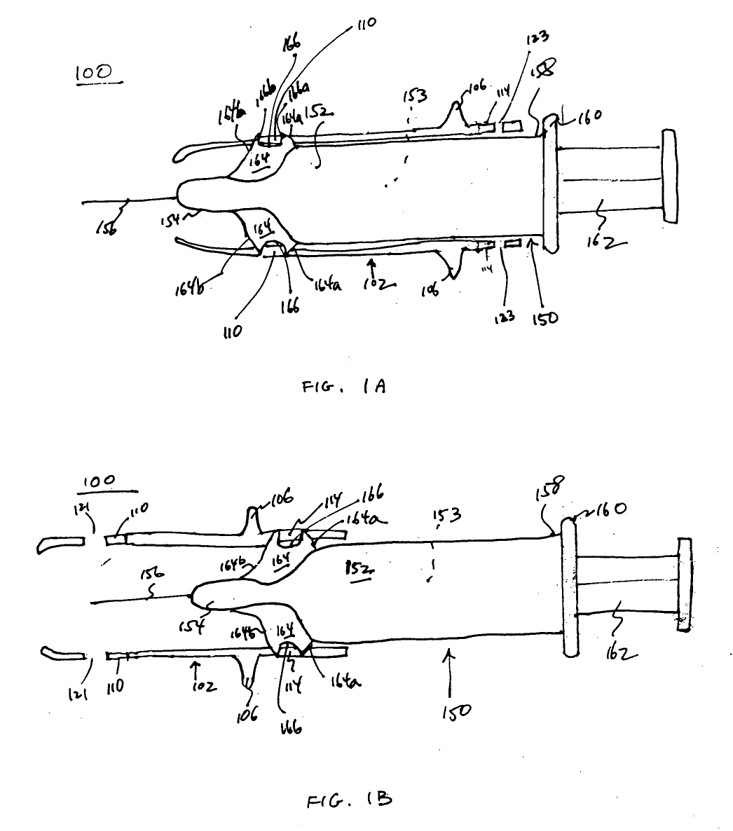

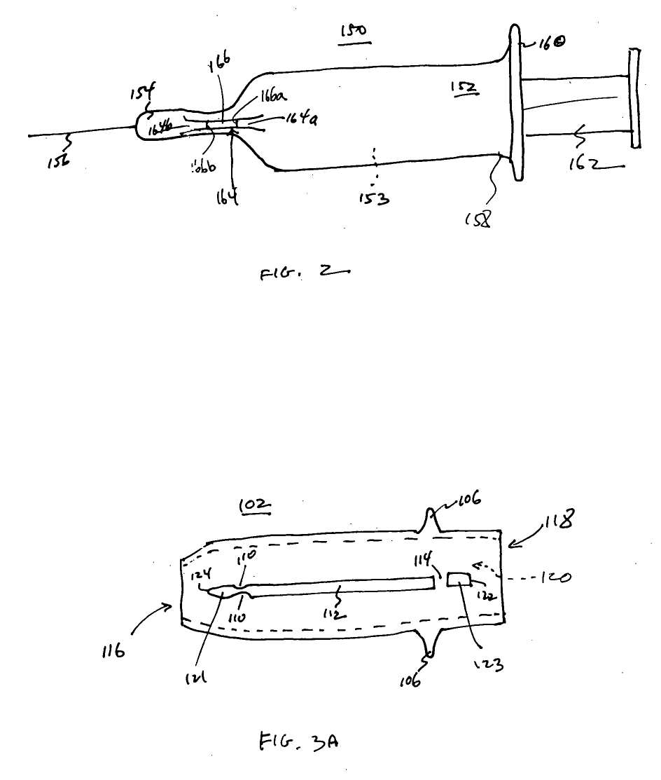

Turning now to the drawings, FIGS. 1A-3C illustrate a first preferred embodiment of an injection device 100 for administering a medical product, e.g., a vaccine, drug, or other therapeutic or diagnostic agent, to a patient, including a syringe 150 and a guard 102, in accordance with the present invention. The guard 102 is slidably attached to the syringe 150 such that the guard 102 may be slidable between a retracted position (shown in FIG. 1A) wherein a needle 156 is exposed, and an extended position (shown in FIG. 1B) wherein the guard 102 extends distally beyond the needle 156.

With particular reference to FIG. 2, the syringe 150 includes an elongate barrel 152 having a distal end or hub 154 from which a needle 156 extends. The needle 156 communicates with an interior lumen or cavity 153 of the barrel 152. In one embodiment, the needle 156 is attached to the hub 154 during manufacturing, and the syringe 150 is provided to a user with the needle 156 pre-attached. Alternatively, the hub 154 includes a connector, e.g., a thread or luer lock adaptor. Here, the syringe 150 may be provided to a user without a pre-attached needle 156, and the user may attach a desired needle 156 to the hub 154 before use.

The syringe 150 also has a proximal end 158 that includes a flange 160. A plunger 162 is inserted into the proximal end 158 for delivering an agent from the interior lumen 153 of the barrel 152 and through the needle 156. In a preferred embodiment, the barrel 152 is generally cylindrical in cross-section. Alternatively, the barrel 152 may have other cross-sectional shapes, e.g., square, rectangular, and the like. The flange 160 may have a sufficiently large width to provide a finger grip for the syringe 150, or may simply be a relatively small lip to facilitate manufacturing, e.g., to facilitate handling on a filling line (not shown).

The syringe 150 further includes one or more lugs 164 that extend transversely or radially outwardly from the barrel 152, e.g., where the barrel 152 narrows to the hub 154 proximal to the needle 156. In one preferred embodiment, a pair of lugs 164 are integrally molded onto an exterior surface of the barrel 152. The lugs 164 are preferably disposed opposite one another on the barrel 152, and may extend axially along a portion of the barrel 152, e.g., generally towards the distal end of the barrel 152. Each of the lugs 164 is substantially rigid, and includes ramped proximal and distal outer edges 164a, 164b. In addition, each of the lugs 164 also includes a notch 166 that defines substantially blunt proximal and distal inner edges 166a, 166b between the proximal and distal outer edges 164a, 164b. Alternatively, the inner edges 166a, 166b may be ramped (not shown) in a manner similar to the outer edges 164a, 164b. The notch 166 on the lug 164 is configured for receiving one or more corresponding first and second guard detents 110, 114 located on the guard 102 (not shown in FIG. 2), as described further below.

In a preferred embodiment, the syringe 150 is manufactured from substantially clear and/or colorless glass, and more preferably from a single piece of glass having the lugs 164 formed integrally thereon. Alternatively, the syringe 150 may be molded as a single integral piece of plastic, e.g., made from polypropylene, k-resin, or polycarbonate, and the like, and including the lugs 164 formed integrally thereon. One suitable process for manufacturing the lugs 164 integral with the syringe 150 is a process using glass forming machines developed by AMBEG GmbH (Berlin, Germany). The fact that the syringe 150 including the lugs 164 is formed as a single piece may be an important feature because it eliminates the need to manufacture and attach separate lugs, detents, or other mechanisms on the syringe before or after receiving the guard thereon, as explained further below. Additionally, with particular respect to the syringe 150 being molded from glass, the configuration and shape of the lugs 164 may reduce the possibility that the lugs 164 or the syringe 150 may be damaged during manufacturing, assembly, and/or handling. For example, the shape and configuration of the lugs 164 may tend to compensate for the generally brittle nature of glass.

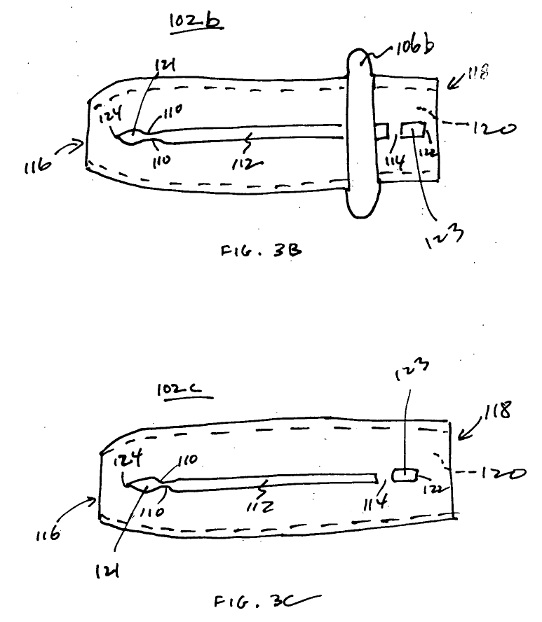

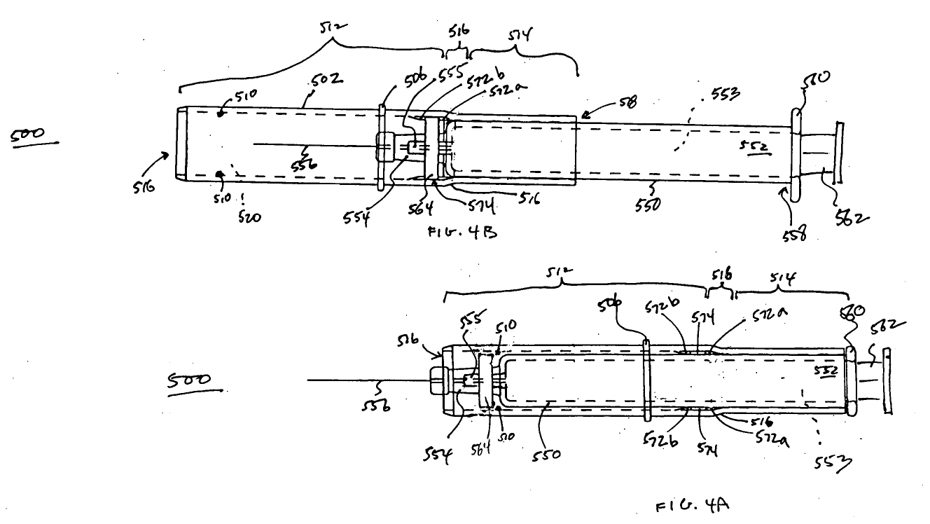

Turning to FIG. 3A, the guard 102 is generally an elongate tubular member including an open proximal end 118, an open distal end 116, and a cavity 120 extending axially between the proximal 118 and distal 116 ends. An inside surface of the cavity 120 has a shape similar to an outer cross-section of the syringe 150. For example, if the outer cross-section of the syringe 150 is cylindrical, the inside surface of the cavity 120 is also cylindrical. Alternatively, if the outer cross-section of the syringe 150 is substantially rectangular or square, the inside surface of the cavity 120 is also substantially rectangular or square.

In the embodiment shown in FIGS. 1A, 1B, and 3A, the guard 102 includes two finger grips or protrusions 106 on opposite sides of the guard 102. The finger grips 106 may facilitate moving the guard 102 relative to the syringe 150. Alternatively, as shown in FIG. 3B, the guard 102b may include an annular finger grip 106b that extends circumferentially around the outer periphery or circumference of the guard 102b. In another alternative embodiment, illustrated in FIG. 3C, a guard 102c may not include finger grips.

Returning to FIG. 3A, the guard 102 includes an elongate opening or slot 112 therethrough, and preferably a pair of opposing slots, that extend axially along the guard 102. The slots 112 may be sufficiently wide to define a window. Additionally, the guard 102 may be formed from substantially clear material to further facilitate observation of the syringe 150 received in the guard 102. The slots 112 preferably have a width similar to a width of the lug 164, thereby providing a traveling slot for the lug 164 of the syringe 150, while limiting rotational movement of the guard 102 around the syringe 150. The slots 112 include proximal and distal edges 122, 124 that may limit axial movement of the guard 102 relative to the syringe 150. The distal edge 124 may limit proximal movement of the guard 102, while the proximal edge 122 may limit distal movement of the guard 102.

First and second detents 110, 114 are provided within the slot 112, adjacent to the distal and proximal edges 124, 122 of the slot 112, respectively. The first detents 110 may be semi-rigid or substantially flexible, and may extend only partially across the slot 112 adjacent the distal edge 124. The second detents 114 are substantially rigid or semi-rigid, and may extend completely across the slot 112 near the proximal edge 122. Additionally, the slot 112 may include a space 123 proximal from the second detents 114 that is configured to receive the ramped proximal outer edge 164a of the lug 164. Also, the slot 112 may include a space 121 distal from the first detents 110, that is configured to receive the ramped distal outer edge 164b of the lug 164.

The guard 102 may be molded from plastic, e.g., polypropylene, k-resin, polycarbonate, and the like, and preferably from a single piece of plastic that includes the first and second detents 110, 114 thereon. In a preferred embodiment, the guard 102 is substantially clear to facilitate observation of the syringe 150 therein. Alternatively, the guard 102 may be translucent or opaque, and/or may be colored.

Returning to FIGS. 1A and 1B, to assemble the injection device 100, the distal end 154 of the syringe 150 (optionally with a needle cover, not shown, covering the needle 156) may be inserted into the proximal end 118 of the guard 102. The guard 102 is placed in an extended position, with the second detents 114 engaging the notches 166 of the lugs 164. Then, due to the deformable nature of the guard 102, i.e., since the guard 102 may be molded from a plastic material, the second detents 114 on the guard 102 may be deflected away from the notches 166 using a distal force on the syringe 150, or a proximal force on the guard 102. In one procedure, a mechanical expander or spreader, such as a speculum, is inserted into the proximal end of the guard 102 and used to deflect the second detents 114 away from the notches 166 on the lugs 164. As the second detents 114 are deflected away from the notches 166, the lugs 164 are allowed to pass under the second detents 114. The lugs 164 may then travel freely along the slot 112 as the syringe 150 is advanced within the cavity 120, until the lugs 164 encounter the first detents 110. Adequate distal force may then be applied to the syringe 150, or proximal force to the guard 102, to cause the ramped distal outer edges 164b to deflect the first detents 110 away from the lugs 164 until the first detents 110 are received in the notches 166, and the ramped distal outer edges 164b rest in space 121. The blunt proximal and distal inner edges 164a, 164b that form the notches 166 may then retain the first detents 110 within the notches 166. Thus, with the first detents 110 engaged within the notches 166 of the lugs 164, the guard 102 may be retained in a retracted position, shown in FIG. 1A, exposing the needle 156 of the syringe 150.

The injection device 110 may then be used to deliver medication, e.g., a vaccine, pharmaceutical, or other therapeutic or diagnostic agent, into a patient. The needle 156 may be inserted into a patient's skin (not shown), and the plunger 162 depressed to inject medication within the barrel 152 via the needle 156 into the patient. Upon completion of the injection, the needle 156 may be withdrawn, and the guard 102 advanced towards the extended position. A distal force may be applied to the guard 102 relative to the syringe 150, thereby causing the first detents 110 to engage the lugs 164 and subsequently be deflected away by the proximal inner edges 166a. Preferably, sufficient force is required such that the first detents 110 are not easily removed from the notches 166 accidentally, yet such that it is not overly difficult to deflect the first detents 110 sufficiently to release the lugs 164 within the slot 112.

After the first detents 110 are removed from the notches 166, the lugs 164 are free to travel along the slot 112 of the guard 102. As the guard 102 is advanced distally, the lugs 164 may pass along the slot 112 until they engage the second detents 114. As further force is applied, the ramped proximal outer edges 164b deflect the second detents 114 outwardly such that the notches 166 may be moved into alignment with the second detents 114. The second detents 114 may then resiliently return inwardly to be received within the notches 166. Because of the increased rigidity of the second detents 114 and/or because of the blunt distal inner edges 166b, the second detents 114 may be substantially secured within the notches 166, thereby substantially locking the guard 102 in the extended position. When the guard 102 is in the extended position, the needle 156 of the syringe 150 is substantially covered, thereby preventing reuse and/or minimizing the risk of accidental needle sticks.

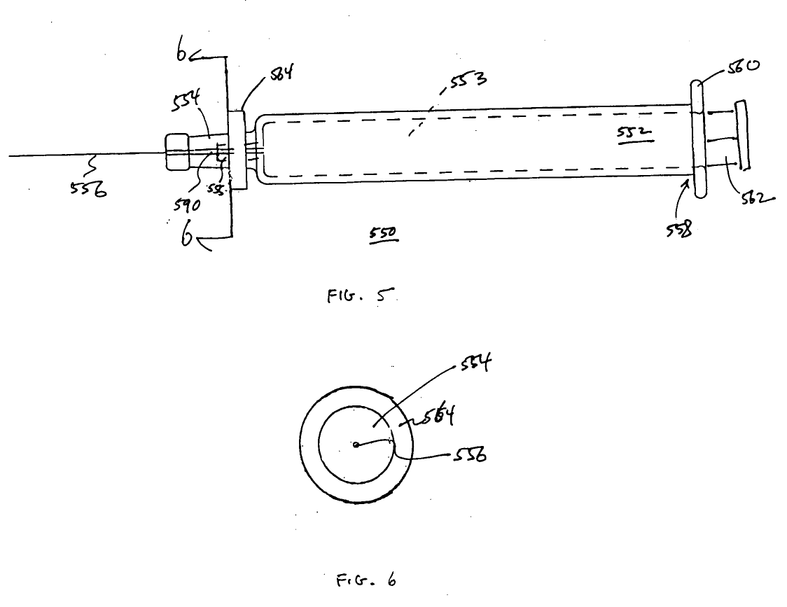

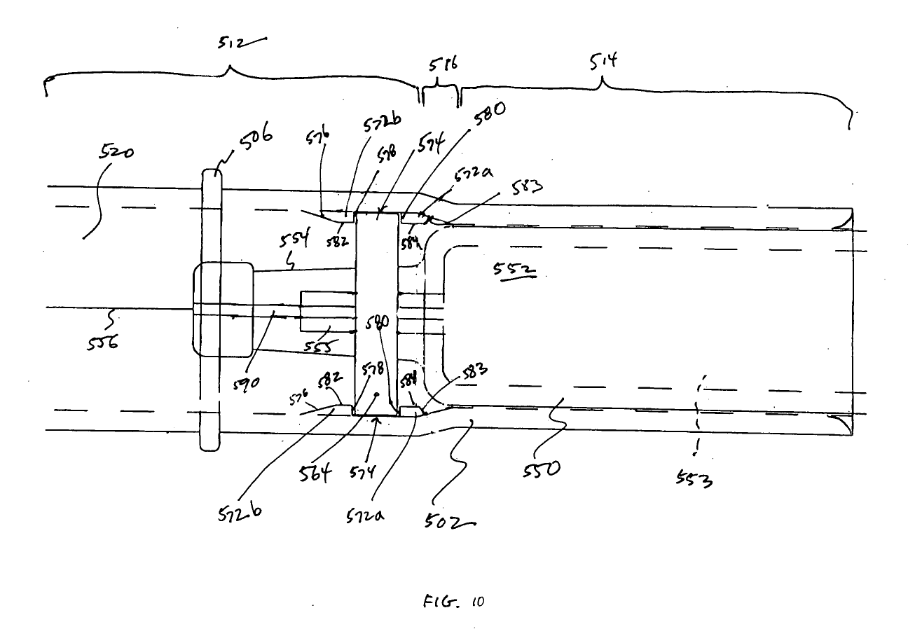

FIGS. 4A-10 illustrate a second preferred embodiment of an injection device 500 for administering medication, e.g., a vaccine, drug, or other therapeutic or diagnostic agent, to a patient, including a syringe 550 and a guard 502, in accordance with the present invention. As with the embodiment of the present invention shown in FIGS. 1-3, the guard 502 of device 500 is slidably attached to the syringe 550. As a result, the guard 502 is slidable between a retracted position (illustrated in FIG. 4B) wherein the needle 556 is exposed, and an extended position (illustrated in FIG. 4A) wherein the guard 502 extends distally beyond the needle 556.

The syringe 550 includes an integrally molded disk 564 for retaining the guard 502 in the retracted and/or the extended position. Like the lugs 164 of syringe 150, the integrally molded disk 564 of syringe 550 may eliminate the need for press fitting a separate disk, lug, detent, detents, or other retaining elements onto the syringe 550 either during manufacture or before use. The disk 564 is preferably of a sufficient thickness that the syringe 550, when formed from glass, may resist damage to the disk 564, notwithstanding the generally brittle nature of glass. Consequently, potential benefits from integrally molding the disk 564 onto the syringe 550 include: reducing tolerance variations, which may reduce the guard 502 failing to be retained in the retracted or extended position due to an improperly fitting disk, and reducing risk of damage to the integrally formed disk during manufacturing, assembling, and/or use.

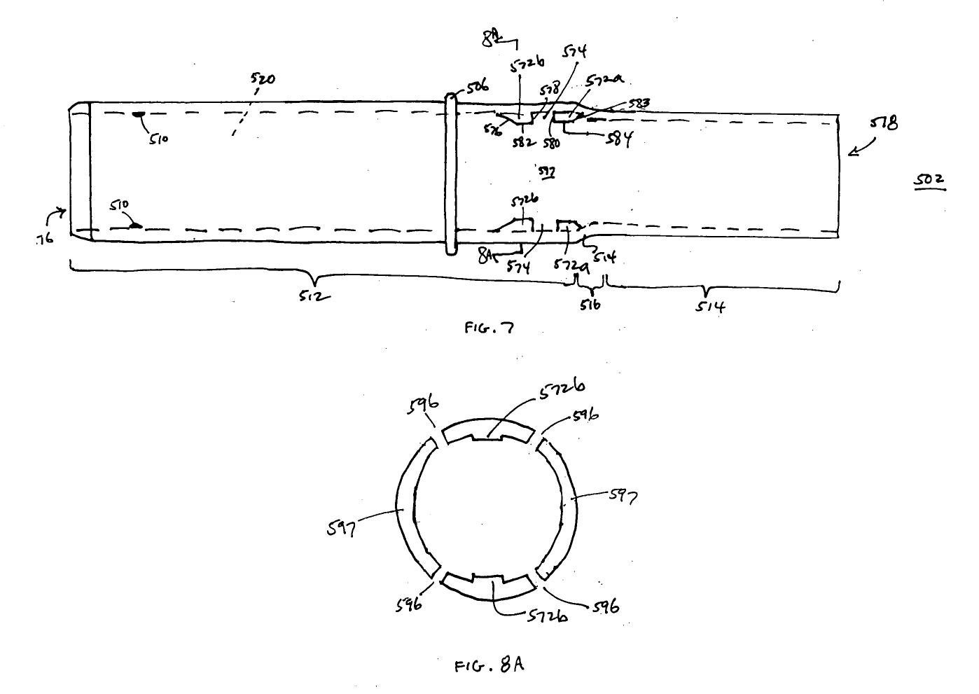

Turning to FIG. 5, the syringe 550 includes an elongate body 552, and a distal end or hub 554 in communication with the body 552 from which the needle 556 extends. In one embodiment, the needle 556 is attached to the hub 554 during manufacturing, and the syringe 550 is provided to a user with the needle 556 pre-attached. In another embodiment, the hub 554 includes a connector, e.g., a thread or luer lock adaptor. Thus, the syringe 550 may be provided to a user without a pre-attached needle 556, and the user may attach a desired needle 556 to the hub 554 before use.

The hub 554 of the syringe 550 illustrated in FIG. 5 may include an intermediate connecting portion 555 between the body 552 and the hub 554 where the needle 556 is attached. The intermediate portion 555 may define a fluid channel 590 that extends from the cavity 553 to the needle 556. Alternatively, no intermediate portion 555 may be required, i.e., the cavity 553 may communicate directly with the needle. Preferably, a plunger 562 is inserted into the proximal end 558 of the syringe 550 for delivering an agent from the lumen 553 of the body 552 and through the needle 156.

The body 552 is preferably generally cylindrical in cross-section. Alternatively, the body 552 may have other cross-sectional shapes, e.g., square, rectangular, and the like. In any event, the body 552 defines a central, interior cavity 553. A therapeutic agent may be contained within the cavity 553 before injecting the agent into a patient. The proximal end 558 may include a flange 560, similar to the previous embodiments.

The syringe 550 further includes an integrally molded disk 564 that extends transversely or radially outwardly from the body 552, e.g., where the body 552 narrows to the hub 554 but proximal from the needle 556. The disk 564 is substantially rigid and is formed from the same material as the rest of the syringe 550, since it is integrally molded as part of the syringe 550. As seen in FIG. 6, the disk 564 has an outer perimeter that extends circumferentially beyond an outer perimeter of the hub 554, and has a diameter that is larger than a diameter of the body 552. As further described herein, the disk 564 may cooperate with detents 510, 572b, 572a to retain the guard 502 in a retracted position and an extended position. Further, the disk 564 may have a width that is substantially the same as a width of a notch 574 defined by detents 572b, 572a.

In a preferred embodiment, the syringe 550 is manufactured from substantially clear glass, and more preferably from a single piece of glass having the disk 564 formed integrally thereon, similar to the syringe 150 described above. Alternatively, the syringe 550 may be molded as a single integral piece of plastic, such as, e.g., polypropylene, k-resin, or polycarbonate, and the like, and including the disk 564 formed integrally thereon. As with the syringe 150, manufacturing the syringe 550 as a single piece including the disk 564 may eliminate the need to manufacture and attach separate lugs, detents, or other mechanisms on the syringe to limit movement of the guard. Furthermore, when the syringe 550 is formed from glass, the disk 564 is preferably of a width that may resist breakage due to the generally brittle nature of glass.

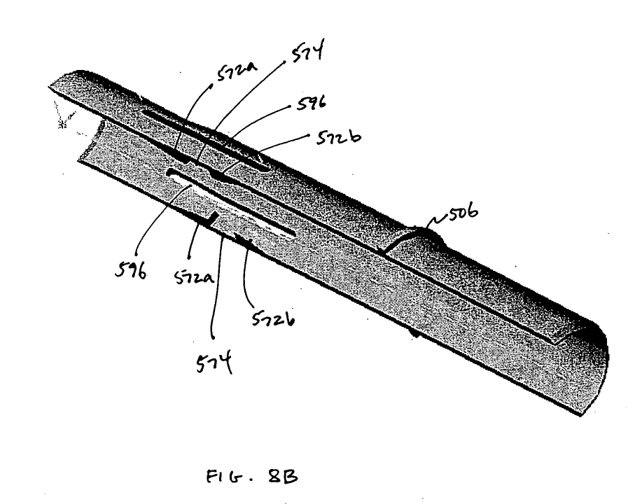

Turning to FIG. 7, the guard 502 is generally an elongate tubular member including an open proximal end 518, an open distal end 516, and a cavity 520 extending axially between the proximal end 518 and distal end 516. The inside surface of the cavity 520 has a shape that conforms to the outer cross-section of the syringe 550. Therefore, if the outer cross-section of the syringe 550 is cylindrical, the inside surface of the cavity 520 is also substantially cylindrical. Alternatively, if the outer cross-section of the syringe 550 is substantially rectangular or square, the inside surface of the cavity 520 is substantially rectangular or square.

The guard 502 may include a distal portion 512 and a proximal portion 514, with a tapering intermediate portion 516 between them. The proximal portion 514 has an inner diameter that is approximately equivalent to the diameter of the body 552 of the syringe 550. The distal portion 512, in comparison, has an inner diameter that is approximately equivalent to the diameter of the disk 564 on the syringe 550, and therefore greater than the proximal portion 514. A tapering intermediate portion 516 connects the distal and proximal portions 512, 514, although the intermediate portion 516 may be blunt (not shown), i.e., directly connecting the proximal and distal portions 514, 512. Since the disk 564 is preferably larger in diameter than the body 552, the intermediate portion 516 tapers, i.e., narrows, toward the proximal portion 514.

As illustrated in FIG. 7, the guard 502 may include a single finger grip 506 that extends circumferentially around the entire outer periphery of the guard 502, although alternatively a pair of finger grips (not shown) may be provided on opposite sides of the guard 502, or the guard 502 may not include any finger grips.

At least one set of proximal detents 572a, 572b may be provided along the inside surface of the guard 502. The detents 572a, 572b may be located distal to the proximal region 514 of the guard 502, i.e., on the inner surface of the distal region 512 of the guard 502. Preferably, between two and four sets of detents 572a, 572b are provided, with the sets being equally spaced around the inside surface of the guard 502. Each set of detents preferably includes first and second ramped detents 572a, 572b. The first ramped detent 572a lies proximal from the second ramped detent 572b. Also, the first detent 572a and the second detent 572b may define a notch 574 that is preferably substantially the same width as the width of the disk 564 of the syringe 550. Consequently, the notch 574 may be configured to accept and retain the disk 564. Each first detent 572a includes a distally facing edge 580 that is oriented toward, and defines the proximal end of, the notch 574. Each first detent 572a further includes a ramped proximal edge 583 that tapers outwardly and toward the proximal portion 514 of the guard 502. The first detent 572a also includes a surface 584 that extends between the distally facing edge 580 and the ramped proximal edge 583. Each second detent 572b includes a proximally facing edge 578 that is oriented toward, and defines the distal end of, the notch 574. Each second detent 572b also includes a ramped leading, or distal, edge 576. The ramped leading or distal edge 576 has a slope that may taper outwardly in a distal direction. A surface 582 may provided between the ramped leading edge 576 and the proximally facing edge 578 that extends substantially parallel to the inside surface wall of the distal portion 512 of the guard 502. The surface 582 preferably lies in the same plane as the proximal portion 514 of the guard 502.

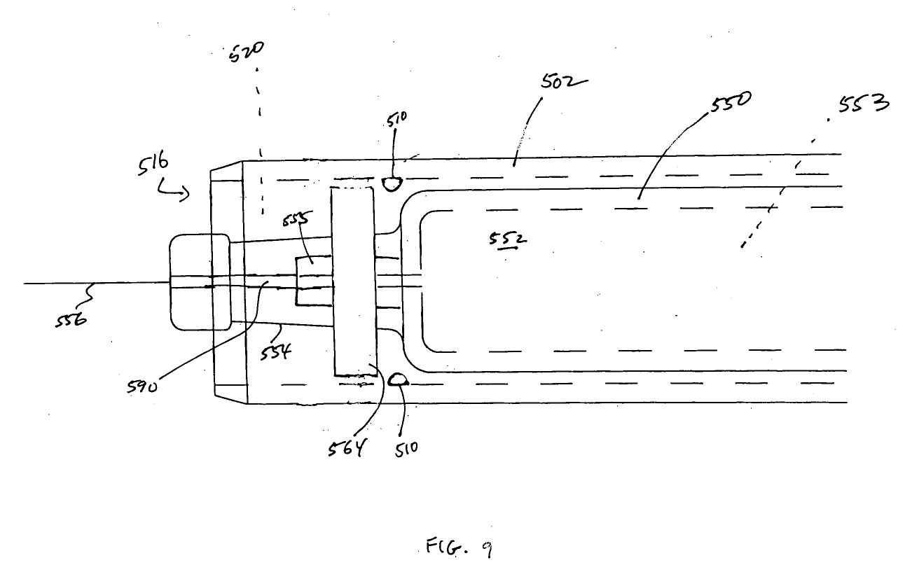

Located adjacent each set of detents 572a, 572b may be a pair of slots 596. In a preferred embodiment, as illustrated in FIG. 7, for example, each slot 596 is a longitudinal slot, and each slot of the pair of slots 596 is separated by, and lies on opposite sides of, a set of detents 572a, 572b. The slots 596 allow for limited expansion of the guard 502 while the disk 564 is travelling over a ramped leading edge 576 of a second detent 572b, or a ramped proximal edge 583 of a first detent 572a, and into the notch 574. Therefore, the slots 596 may facilitate movement of the guard 502 into the extended and retracted positions. A non-flexing side wall 597 between two slots 596 increases the structural integrity of the guard 502. FIG. 8B illustrates a perspective, cross-sectional view of the arrangement of slots 596 adjacent detents 572a, 572b. FIG. 8A is a cross-sectional view of the guard 502 taken along the line 8A-8A. One preferred configuration of second detents 572b, slots 596, and side walls 597 is shown. The embodiment shown in FIG. 8A includes two sets of detents 572b with two sets of slots 596, each set of slots 596 lying on opposite sides of a detent 572b, and a side wall 597 between two slots 596.

Returning to FIG. 7, distal detents 510 may be provided near the distal end 516 of the guard 502. The distal detents 510 may be formed on the inner surface of the guard 502. The distal detents 510 are also preferably offset from the distal end 516. Additionally, in the illustrated embodiment, the distal detents 510 project slightly into the cavity 520 of the guard 502, thereby reducing the diameter of the cavity 520 at the location of the distal detents 510. Preferably, the diameter of the cavity 520 at the location of the distal detents 510 is marginally smaller than the diameter of the disk 564 of the syringe. As a result, the syringe 550 may be advanced such that the disk 564 abuts and comes into contact with the distal detents 510. The syringe 550 may be forcibly engaged with the distal detents 510, and the disk 564 advanced distally past the distal detents 510, such that the distal detents 510 and the disk 564 aid in retaining the guard 502 in the retracted position. In an alternative embodiment, the distal detents 510 may be one annular detent extending entirely around the inner diameter of the guard 502.

The guard 502 may be molded from plastic, such as polypropylene, k-resin, or polycarbonate, and the like. Preferably, the guard 502 is molded from a material that allows for deformation of the guard 502 when the syringe 550 is moved within the guard 502, such as when the disk 564 is advanced past the first and second detents 572a, 572b, or the distal detents 510. The guard 502 is preferably substantially clear to facilitate observation of the syringe 550 therein. Alternatively, the guard 502 may be translucent or opaque, and/or may be colored.

Turning again to FIGS. 4A and 4B, the injection device 500 is assembled by inserting the distal hub 554 of the syringe 550 (optionally with a needle cover, not shown, covering the needle 556) into the proximal end 518 of the guard 502. The guard 502 is then moved proximally relative to the syringe 550, and the proximal portion 514 deforms slightly as the guard 502 is moved over the syringe 550. This allows the proximal portion 514 of the guard 502 to pass over the disk 564 of the syringe 550. After the proximal portion 514 of the guard passes over the disk 564, the disk 564 and the notch 574 formed by first and second detents 572a, 572b will engage. At this stage, the guard 502 is in an extended position. To further advance the guard 502 proximally, the user exerts sufficient proximal pressure on the guard 502 such that the disk 564 slightly deforms the distal portion 512 of the guard 502, substantially at the second detent 576b, and the disk 564 and the first and second detents 576a, 576b disengage. In one procedure, a mechanical spreader or expander, such as a speculum, is inserted into the distal portion 512 of the guard 502, and used to expand the distal portion 512, while the guard 502 is being further advanced proximally. The guard 502 is advanced proximally until the disk 564 of the syringe 550 contacts the distal detents 510 of the guard 502. As previously discussed, the distal detents 510 project into the cavity 520 of the guard 502, thereby reducing the inner diameter of the cavity 520 to a size marginally less than the diameter of the disk 564. After the disk 564 abuts the distal detents 510, additional proximal force is applied to the guard 502, such that the disk 564 deflects the distal detents 510. The guard 502 is then advanced until the distal detents 510 are in position to secure the disk 564, such as by being positioned proximally from the disk 564, and therefore retain the guard 502 in a retracted position relative to the syringe 550.

With the guard 502 is in a retracted position, the needle 556 may be exposed. The injection device 500 may then be used to deliver medication, e.g., a vaccine, pharmaceutical or other therapeutic or diagnostic agent, into a patient. The needle 556 may be inserted into a patient's skin (not shown), and the plunger 562 depressed to inject medication within the body 552 via the needle 556 into the patient. Upon completion of the injection, the needle 556 may be withdrawn, and the guard 502 advanced toward the extended position.

The guard 502 may be advanced toward the extended position by applying a distal force to the guard 502 to disengage the disk 564 from the distal detents 510. Once disengaged, additional force may be applied to advance the guard 502 distally toward the extended position. As the guard 502 is advanced toward the extended position, the disk 564 may contact the second detents 572b. More particularly, the disk 564 initially may contact the ramped leading edges 576 of the second detents 572b. After the disk 564 of the syringe 550 contacts the ramped leading edge 576, additional force is applied in order to deflect the second detents 572b, and consequently the guard 502, outwardly away from the syringe 550. The guard 502 may be advanced distally until the disk 564 is retained by notch 574. Once the disk 564 of the syringe 550 is retained by notch 574, further movement of the guard 502, either toward the retracted position or further distally, is prevented by the proximally facing edge 578 of the second detent 572b and the distally facing edge 580 of the first detent 572a, respectively. Here, the guard 502 is in the extended position. With the guard 502 extended, the needle 556 of the syringe 550 is substantially covered, thereby preventing reuse and/or minimizing the risk of accidental needle sticks.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

1. An injection device, comprising:
a glass syringe comprising a body including proximal and distal ends, the body defining a cavity for receiving a medical product therein;
a glass disk integrally molded to the syringe and extending radially outwardly from the distal end of the syringe;
a guard slidably attached to the syringe comprising a distal portion and a proximal portion having a size for sliding along the body of the syringe, the guard being slidable between a first position wherein the distal end of the syringe is exposed and a second position wherein the distal end of the syringe is covered by the guard; and
detents on the guard for selectively engaging the disk for retaining the guard in at least one of the first and second positions.

2. The injection device of claim 1, wherein the disk has a diameter, the body of the syringe has a diameter, the diameter of the disk being larger than the diameter of the body of the syringe.

3. The injection device of claim 1, wherein the distal portion of the guard has an inner diameter greater than a diameter of the disk, the proximal portion of the guard has an inner diameter less than the diameter of the disk, the guard further comprising an intermediate portion that tapers from the distal portion to the proximal portion.

4. The injection device of claim 1, wherein the detents comprise a distal detent on an inner surface of the distal portion of the guard, the distal detent configured for engaging the disk to retain the guard in the first position.

5. The injection device of claim 4, wherein the detents comprise a proximal detent on an inner surface of the guard proximal to the distal detent, the proximal detent configured for engaging the disk to retain the guard in the second position.

6. The injection device of claim 5, wherein the proximal detent comprises a blunt proximal edge for engaging the disk, thereby preventing subsequent movement of the guard from the second position towards the first position.

7. The injection device of claim 1 further comprising a needle extending from the distal end of the syringe.

8. The injection device of claim 11 further comprising a hub on the distal end of the syringe, and a needle coupled to the hub.

9. An injection device, comprising:
a glass syringe comprising a body having a proximal end, a distal end, a plunger extending from the proximal end, and a hub on the distal end;
a glass disk integrally molded to the syringe and extending radially outwardly from the hub;
a guard slidably attached to the syringe comprising a distal portion and a proximal portion having a size for sliding along the body of the syringe, the guard being slidable between a first position and a second position;
a first detent on an inner surface of the guard for engaging the disk to retain the guard in the first position; and
a second detent on an inner surface of the guard for engaging the disk to retain the guard in the second position.

10. The injection device of claim 9 further comprising a needle coupled to the hub.

11. The injection device of claim 9 further comprising a connector on the hub and a needle attached to the connector.

12. The injection device of claim 9, wherein the distal end of the syringe is exposed in the first position, and covered in the second position.
